(19) United States
(12) Patent Application Publication
(10) Pub. No.: US 2007/0093760 A1
(43) Pub. Date: Apr. 26, 2007

(54) Syringe with wound dressing material

(76) Inventor: Michael R. Wexler, St. Paul, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/247,824

(22) Filed: Oct. 11, 2005

(51) Int. Cl. A61M 5/00

(52) U.S. Cl. 604/187

(57) ABSTRACT

Disclosed is a syringe including a wound dressing for arresting bleeding and/or providing a barrier against microorganisms at a needle insertion point on the skin of a patient. The wound dressing is releasably integrated with the syringe. In some embodiments, the syringe includes a bandage disposed on the posterior end of the needle hub of the syringe such that the needle extends through the bandage. In other embodiments the syringe includes a reservoir containing a dressing substance. The dressing may be adhered, or otherwise applied, to the skin of the patient proximate the needle insertion point prior to withdrawing the needle of the syringe from the patient. As the needle is withdrawn, the dressing remains at the insertion point on the skin of the patient to arrest bleeding and/or provide a barrier against microorganisms at the wound site.

### TECHNICAL FIELD

The present invention generally relates to syringes. More particularly, the present invention relates to a wound dressing integrated with a hypodermic syringe intended for applying a dressing on the skin of a patient at a needle insertion point upon withdrawal of the syringe from a patient.

### BACKGROUND

Syringes are commonly used by medical personnel and individuals to administer injections of medicinal fluids and letting of fluid samples from a body. Syringes may be used for various forms of intravenous, subcutaneous, and intramuscular injection, or for the removal of blood (i.e., venipuncture) or other bodily fluids. It has been estimated that ten million injections are given daily in the United States, with annual usage of syringes exceeding twelve billion injections worldwide.

Direct contact with blood or other bodily fluids poses significant health risks to health care workers and others. Many pathogens, such as HIV, AIDS, Hepatitis B, Hepatitis C, as well as other bloodborne pathogens, are transmitted through blood or other bodily fluids. It is estimated that over 12,000 health care workers are infected with diseases through inadvertent contact with blood each year.

There is a significant risk of contracting a disease from the use of a syringe. As a needle is withdrawn from a patient, there is a natural tendency for bleeding to occur at the needle insertion point through the skin of the patient until hemostasis. Typically, a health care worker or user disposes of the syringe and then applies a bandage to the wound. Thus, an additional step is required after withdrawing the needle in order to apply a bandage to the wound. Pressure from the bandage slows bleeding from the wound and facilitates hemostasis. In applying the bandage, the health care worker or user typically must bring his/her hands and/or other appendages in close proximity to the wound, thus increasing the risk of inadvertently coming into contact with the patient's blood or other bodily fluid.

It would be desirable to provide a syringe that may apply a dressing to a needle insertion point on the skin of a patient without the need of the health care worker or user directly contacting the bandage and/or the area proximate the needle insertion point. It would be desirable to provide a syringe that may apply a dressing to a needle insertion point on the skin of a patient upon withdrawing the needle from the patient.

### SUMMARY

The invention is directed to a syringe including a dressing, or other wound covering matter, for arresting bleeding and/or providing a barrier against microorganisms at a needle insertion point on the skin of a patient. In some embodiments, the dressing may be releasably incorporated or integrated with the syringe.

Accordingly, one embodiment is a syringe having a bandage disposed thereon. The bandage may be disposed on the posterior end of the needle hub such that the needle extends through the bandage. The bandage may be adhered, or otherwise applied, to the skin of the patient proximate the needle insertion point prior to withdrawing the needle of the syringe from the patient. As the needle is withdrawn, the bandage remains at the insertion point on the skin of the patient to arrest bleeding and/or provide a barrier against microorganisms at the wound site.

Another embodiment is a syringe including a reservoir containing a dressing substance. While administering an injection, but prior to or during withdrawal of a needle from the patient, the dressing material may be expelled from the syringe at the needle insertion point on the skin of the patient. As the needle is withdrawn, the dressing substance remains at the insertion point on the skin of the patient to arrest bleeding and/or provide a barrier against microorganisms at the wound site.

### BRIEF DESCRIPTION OF THE DRAWINGS

The invention may be more completely understood in consideration of the following detailed description of various embodiments in connection with the accompanying drawings, in which:

FIG. 1 is a partially cross-sectioned side view of an illustrative syringe;

FIG. 2 is a cross-sectional view of a portion of the syringe of FIG. 1;

FIG. 3 is a cross-sectional view of a portion of another illustrative syringe;

FIG. 4 is a top view of the bandage shown with the syringe in FIG. 3;

FIG. 5 is a side view of the bandage shown with the syringe in FIG. 3;

FIG. 6 is a perspective view of an illustrative syringe including a bandage;

FIG. 7 is a perspective view of an illustrative syringe including a bandage;

FIG. 8 is a perspective view of an illustrative syringe including an exemplary safety needle guard;

FIG. 9 is a cross-sectional view of an illustrative cap for a syringe needle including a bandage;

FIG. 10 is a cross-sectional view of an illustrative cartridge including a bandage for a syringe;

FIG. 11 is a cross-sectional view of another illustrative cartridge including a bandage for a syringe;

FIG. 12 is a perspective view of a panel of bandages for distributing a bandage onto a syringe; and

FIG. 13 is a cross-sectional view of another illustrative syringe.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the term “posterior” and its derivatives is intended to refer to the orientation of components in the direction toward the tip of the needle of a syringe. As used in this specification and the appended claims, the term “anterior” and its derivatives is intended to refer to the orientation of components in the direction opposite the tip of the needle of a syringe.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

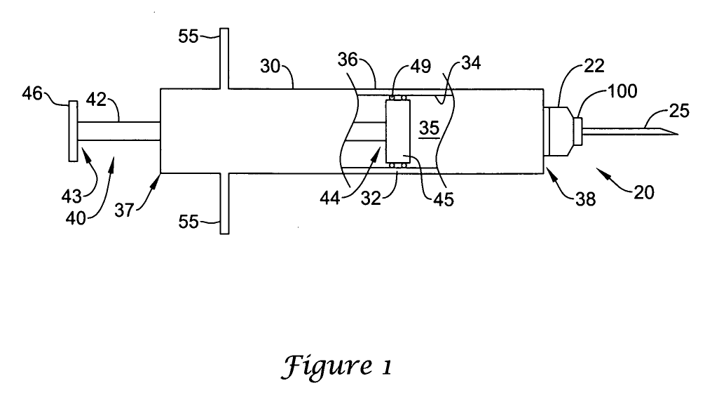

Now referring to FIG. 1, an illustrative syringe 10 is shown. The syringe 10 may be a conventional syringe, or the syringe 10 may be a safety syringe wherein the needle 25 is shrouded by a structure in order to deter inadvertent contact with the needle 25 and/or fluids on or in the needle 25 following administration of an injection or letting of a bodily fluid. For example, in some embodiments, the needle 25 may automatically or manually retract into the chamber 35 of the barrel 30 of the syringe 10 by activating a spring mechanism (not shown), or by engaging the plunger 40 with the needle assembly 20 and drawing the plunger 40 in an opposite direction, thereby retracting the needle 25 into the chamber 35 of the barrel 30. Thus, the barrel 30 may act as a container encapsulating the needle 25 in order to prevent unintentional contact. In other embodiments, a cover or shield (not shown) may be positioned over or around the needle 20 to shroud the needle 25 in order to prevent unintentional contact with the needle 25, such as unintentional needle pricks, and/or inadvertent contact with fluids on or in the needle 25. Other embodiments may utilize other mechanisms and/or structures for shrouding or otherwise deterring inadvertent contact with the needle 25.

The syringe 10 may include a barrel 30 having a tubular wall 32. The tubular wall 32 includes an inner surface 34 defining a chamber 35 and an outer surface 36. The chamber 35 may form a hollow compartment for containing a fluid. The barrel 30 also includes a first end 37 and a second end 38. In some embodiments, the first end 37 may be an open end for receiving a plunger 40. The second end 38 may be configured to receive and/or be coupled to a needle assembly 20 including a needle 25. In some embodiments, the second end 38 may be a reduced diameter portion. For example, the second end 38 may include a frusto-conical tip configured to receive and frictionally engage a frusto-conical portion of a needle assembly 20. In other embodiments, the second end 38 may include a threaded portion, such as a male threaded portion or a female threaded portion, for threadably engaging a complementary threaded portion, such as a female threaded portion or a male threaded portion, of a hub assembly 20. A needle assembly 20 may also be coupled to the second end 38 of the barrel 30 by adhesive, thermal bonding, molding, mechanical interlocking means, frictional engagement, or other attachment means generally known within the art.

The plunger 40 may be slidably positioned in fluid tight engagement with the inner surface 34 of the barrel 30. In some embodiments, the plunger 40 includes an elongate plunger rod 42 having a first end 43 and a second end 44. The plunger 40 may include an actuator 46 at the first end 43 which may be used as a thumbrest for facilitating a user in actuating the plunger 40. The plunger 40 may include a piston 45 at the second end 44. The piston 45 may include one or more resilient sealing members such as annular seals 49 for providing fluid tight engagement with the inner surface 34 of the barrel 30. The plunger 40 may be longitudinally actuated such that the piston 45 slidably moves throughout at least a portion of the length of the chamber 35. Thus, actuation of the plunger 40 may push the piston 45 toward the needle 25, expelling fluid out of the chamber 35 of the syringe 10 through the needle 25, and/or actuation of the plunger 40 may pull the piston 45 away from the needle 25, drawing fluid into the chamber 35 of the syringe 10 through the needle 25.

In some embodiments, the barrel 30 may include one or more tabs 55 extending outward from the outer surface 36 of the barrel 30. The one or more tabs 55 may be positioned at any desired location along the outer surface 36 of the barrel 30. For example, the one or more tabs 55 may be located proximate the first end 37 of the barrel 30. The one or more tabs 55 may be finger grips, such that a user may hold the syringe 10 in one hand with a thumb resting on the actuator 45 and two fingers resting on the tabs 55. Additionally, the one or more tabs 55 and/or the actuator 45 may assist the user in providing an axial force to actuate the plunger 40 through the barrel 30.

The needle assembly 20 may include a needle hub 22 and a needle 25. The needle 25 may be any desired size. In some embodiments, the needle 25 may be a gauge 32 to a gauge 8 needle, although the needle 25 may be of a smaller or larger gauge in other embodiments. The needle 25 includes an inner lumen (not shown) in fluid communication with the chamber 35 of the barrel 30. The needle hub 22 may couple the needle 25 to the barrel 30 of the syringe 10. The needle hub 22 may be a unitary part of the needle 25, or the needle hub 22 may be a separate component of the needle assembly 20 configured to secure the needle 25 to the syringe 10. Thus, in some embodiments, the needle assembly 20 may be a removable component of the syringe 10 removable from the barrel 30, and in other embodiments, the needle assembly 20 may be a fixed component of the syringe 10 permanently secured to the barrel 30. In some embodiments, the needle hub 22 may include a threaded portion, such as a female threaded portion or a male threaded portion, configured to threadably engage a threaded portion, such as a male threaded portion or a female threaded portion, extending from the barrel 30. In other embodiments, the needle hub 22 may be frictionally engaged with a portion of the syringe 10 extending from the barrel 30. The needle assembly 20 may also be coupled to the second end 38 of the barrel 30 by adhesive, thermal bonding, molding, mechanical interlocking means, frictional engagement, or other attachment means generally known within the art.

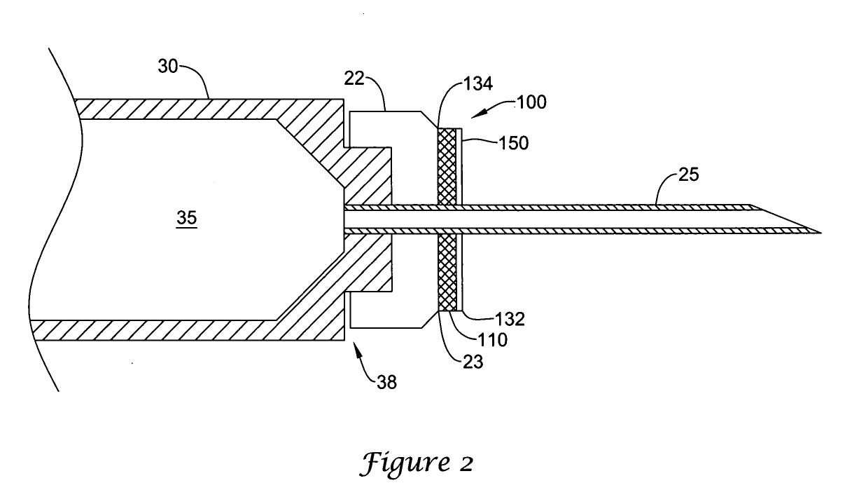

A wound dressing, such as bandage 100, may be positioned on the syringe 10. In some embodiments, the bandage 100 may be releasably incorporated or integrated with the syringe 10. The bandage 100 may include an adhesive material, an absorbent material, an elastomeric material, a self-sealing material, a fibrous material, a gelatin substance, a liquidous substance, an ointment, or any combination or laminate of the same. As shown in FIG. 2, the bandage 100 may be positioned on the posterior end 23 of the needle hub 22 such that the needle 25 extends through the bandage 100, for example. In some embodiments, the bandage 100 may be cylindrical. However, the bandage 100 may be any desired shape. In some embodiments, the bandage 100 may include an elastomeric, self-sealing membrane 110, or other layer of material which may provide a radially inward force to sufficiently seal, collapse, enclose, or otherwise obstruct the opening through which a needle 25 may extend through the bandage 100. Thus, blood may not be able to traverse the bandage 100. Additionally or alternatively, the bandage 100 may include a layer of gauze, cotton, or other absorbent material. A first surface 132 of the bandage 100 may include an adhesive coating 150 for adhering the bandage 100 to the skin of a patient. The adhesive coating 150 may have a strong enough affinity to the skin of a patient to retain the bandage 100 at the insertion point of the needle 25 through the skin of a patient when the needle 25 is withdrawn from the patient. Thus, the bandage 100 may be adhered to the skin of the patient prior to withdrawing the needle 25, such that the bandage 100 remains adhered to the skin of the patient proximate the needle insertion point upon withdrawal of the needle 25. In some embodiments, the second surface 134 of the bandage 100 may abut, be releasably coupled to, and/or be temporarily secured to the posterior end 23 of the needle hub 22.

During administration of an injection or letting of a bodily fluid, the needle 25 of the syringe 10 first pierces the skin of the patient and then extends into tissue of the patient. The location where the needle 25 penetrates the epidermis of the patient is described herein as the needle insertion point. This is the location bleeding will occur when the needle 25 is withdrawn. The plunger 40 of the syringe 10 may be actuated anteriorly to withdraw bodily fluid from the patient into the chamber 35 and/or posteriorly to expel fluid from the chamber 35 into the body of the patient. Prior to withdrawing the needle 25 from the patient, the bandage 100 is adhered to, or is otherwise applied to, the skin of the patient proximate the needle insertion point. Thereafter, when the needle 25 is withdrawn from the patient, the bandage 100 remains at the needle insertion point to arrest bleeding and/or provide a barrier against microorganisms at the wound site. One or more of the materials forming the bandage 100 may sufficiently seal, collapse, enclose, or otherwise obstruct the opening formed in the bandage 100 in which the needle 25 extends through when the needle 25 is withdrawn from the patient and thus withdrawn from the bandage 100. In some embodiments, one or more of the materials forming the bandage 100 may provide a radially inward force in order to sufficiently seal, collapse, enclose, or otherwise obstruct the opening formed in the bandage 100 from insertion of a needle 25 therethrough.

Thus, the health care worker or user need not apply a bandage to the wound during an additional step after withdrawing the needle 25. Furthermore, the health care worker or user need not bring his/her appendages or other body parts into close proximity with the needle insertion point, thus reducing the likelihood of inadvertently contacting blood or other bodily fluids. Additionally, by applying the bandage 100 to the needle insertion point prior to withdrawing the needle 25 from the skin, blood escaping from the needle insertion point may be reduced or eliminated. The process may also be less traumatic and more efficient that prior techniques.

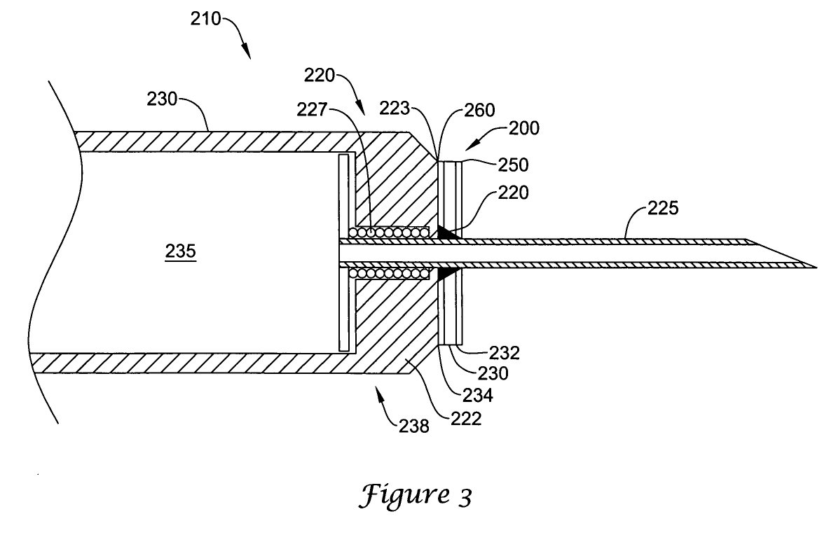

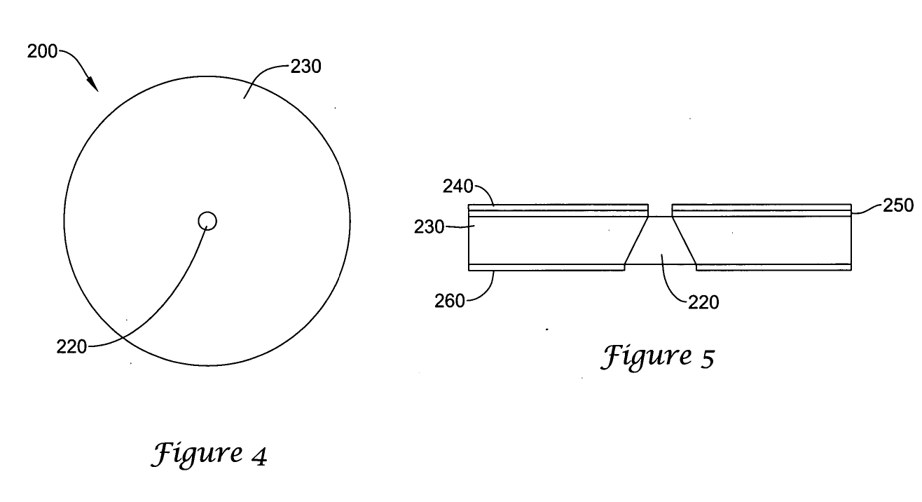

Another illustrative wound dressing, bandage 200, is shown in FIGS. 3-5. The syringe 210 shown in FIG. 3 may include a retractable needle 225 which may be retracted into the chamber 235 of the barrel 230 of the syringe 210. In some embodiments, the hub assembly 220 at the end 238 of the syringe 210 may be a unitary component of the barrel 230 of the syringe 210. The hub assembly 220 may include a spring 227, a vacuum, or other mechanism which may retract the needle 225 into the chamber 235 of the barrel 230.

The bandage 200 may be releasably incorporated or integrated with the syringe 210. In some embodiments, the bandage 200 may abut or otherwise be disposed proximate the posterior end 223 of the needle hub 222. The bandage 200 may be a circular disk having a center portion 220 and an outer annular portion 230 surrounding the center portion 220. The outer annular portion 230 may include a flexible material, such as an elastomeric fiber material, a self-sealing material, or the like. The center portion 220 may be configured to receive the needle 225 of the syringe 210. In some embodiments, the center portion 220 may include an absorbent material, such as gauze or cotton, or another material, such as a gelatin material, an ointment, a laminate, or other material. In some embodiments, the center portion 220 may include a through opening extending through the bandage 200 in which the needle 225 may extend through. A material disposed in the center portion 220 may sufficiently seal, collapse, enclose, or otherwise obstruct the opening formed through the center portion 229 of the bandage 200 in which the needle 225 extends through when the needle 225 is withdrawn from the patient and thus withdrawn from the bandage 200. For example, in some embodiments, an absorbent material may be compressably packed in the center portion 220 around the needle 225. Once the bandage 200 is isolated from the needle 225, the absorbent material in the center portion 220 may expand, swell, shift, or otherwise or at least partially fill the opening formed by the needle 225. Thus, a hole or opening formed in the bandage 200 when the needle 225 extends through the bandage 200 may be sufficiently sealed, collapsed, enclosed, or otherwise obstructed when the needle 225 is separated from the bandage 200. In some embodiments, the needle 225 may extend through the center portion 220 of the bandage 200. Therefore, the center portion 220 may provide relatively minimal frictional contact with the needle 225 in embodiments wherein the needle 225 is configured to retract into the barrel 230 of the syringe 210. The center portion 220 may be centered over a needle insertion point through the skin of a patient, thus absorbing blood egressing through the needle insertion point when the needle 225 is withdrawn from the patient. In embodiments wherein the center portion 220 includes an absorbent material, the absorbent material may obstruct the central opening through the bandage 200 subsequent separation of the needle 225 from the bandage 200.

The annular portion 230 may include a first surface 232 configured to be placed in contact with the skin of a patient. The first surface 232 may include an adhesive coating 250 for adhering the bandage 200 to the skin of a patient. Prior to use, the first surface 232 may be covered by a covering 240, as shown in FIG. 5. The covering 240 may protect the adhesive coating 250 and/or preserve the sterile environment of the bandage 200. The covering 240 may have a weak bonding affinity to the adhesive coating 250, thus the covering 240 may be readily removed from the bandage 200 as desired without displacing the bandage 200 on the syringe 210. For example, the covering 240 may have a waxy surface which may not adhere well to the adhesive coating 250. In some embodiments, the second surface 234 opposite the first surface 232 of the bandage 200 may include an adhesive coating 260 for temporarily securing the bandage 200 to the posterior end 223 of the needle.hub 222. The adhesive coating 250 may provide a stronger affinity to the skin of a patient than the affinity of the adhesive coating 260 to the posterior end 223 of the needle hub 222. Thus, when the bandage 200 is adhered to the skin of the patient, the bandage 200 may be released from the posterior end 223 of the needle hub 222. In other words, the bond between the adhesive coating 250 and the skin of a patient may be stronger than the bond between the adhesive coating 260 and the posterior end 223 of the needle hub 222.

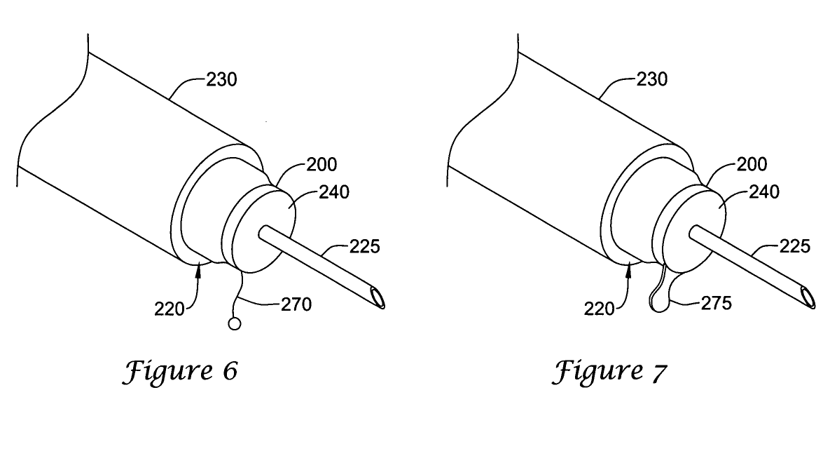

As shown in FIG. 6, the covering 240 may include a filament 270, such as a string, wire, cord, thread, or the like, to facilitate removal of the covering 240 prior to use. As the covering 240 may be quite small in some embodiments, a health care worker or other user may more easily and efficiently grasp the filament 270 to remove the covering 240 from the bandage 200. Additionally, removing the covering 240 with the filament 270 may not contaminate the sterile environment of the syringe 210 desired in medical procedures.

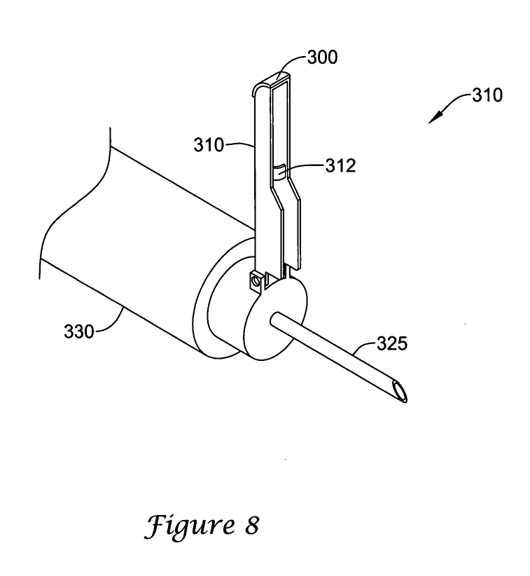

Alternatively, as shown in FIG. 7, the covering 240 may include a tab 275. In some embodiments, the tab 275 may be a unitary or integral portion of the covering 240. However, in other embodiments, the tab 275 may be a discrete component of the bandage 200. The tab 275 may extend beyond other portions of the bandage 200, such that a health care worker or other user may readily grasp the tab 275 in order to remove the covering 240 from the bandage 200. As the covering 240 may be quite small in some embodiments, a health care worker or other user may more easily and efficiently grasp the tab 275 to remove the covering 240 from the bandage 200.

FIG. 8 shows a syringe 310 having a needle guard 314 attached thereto. The needle guard 314 may be hingedly attached to the barrel 330 of the syringe 310, slidably attached to the barrel 330 of the syringe 310, or otherwise attached to the syringe 310 such that after withdrawal of the needle 325 from the skin of a patient, the needle guard 314 may be repositioned over the needle 325 to encapsulate the needle 325. The needle guard 314 may be configured such that the needle 325 cannot be reversibly exposed from the needle guard 314 once the needle guard 314 has been positioned over the needle 325 to encapsulate the needle 325. For example, the needle guard 314 may include one or more locking tabs 312 such that the needle 325 becomes engaged with the one or more locking tabs 312 when the needle guard 314 is repositioned over the needle 325 in order to prevent the needle 325 from being exposed from the needle guard 314.

The needle guard 314 may include a wound dressing, such as a bandage 300, releasably attached thereon. The bandage 300 may include an adhesive coating 350 for adhering the bandage 300 to the skin of a patient. Thus, upon withdrawal of the needle 325 from the skin of a patient, the needle guard 310 may be positioned over the needle 325. Simultaneously or in a subsequent step, the bandage 300 may be applied to the needle insertion point on the skin of the patient. Thus, the bandage 300 may be applied to the needle insertion point without the health care worker or other user having to come into contact with the skin of the patient. The bandage 300 may include a covering (not shown) which extends over and protects the adhesive coating 350 prior to use.

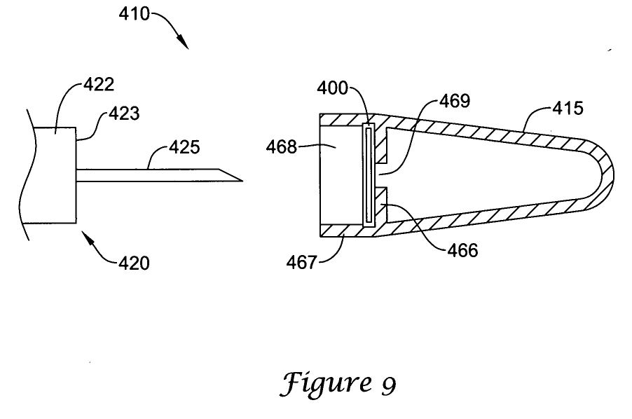

FIG. 9 shows a cap 465 for a syringe 410. The cap 465 may be configured to receive a needle 425 of a needle hub assembly 420 of the syringe 410. In some embodiments, the cap 465 may include a base portion 467 configured for engaging the hub 422 of the needle hub assembly 420. For example, in some embodiments, the base portion 467 may frictionally engage with the hub 422, the base portion 467 may mechanically interlock with the hub 422, or the base portion 467 may include a threaded portion configured to threadably engage with a complementary threaded portion of the hub 422. In other embodiments, the base portion 467 of the cap 465 may be sized larger than the hub 422 such that the cap 465 fits loosely over the hub 422, or the base portion 467 of the cap 465 may be sized smaller than the hub 422 such that the cap 465 may not fit over the hub 422.

A wound dressing, such as a bandage 400, may be positioned in the cavity 468 of the base portion 467 of the cap 465. The bandage 400 may be substantially similar to other bandages previously disclosed herein, or the bandage 400 may be dissimilar. The bandage 400 may include a central portion or core including an absorbent material, and an outer portion surrounding at least a portion of the central portion which may include an elastomeric fibrous material and/or an adhesive material, for example. The bandage 400 may abut, adhere to, engage with, or otherwise be disposed within a recess portion 466 within the cap 465. In some embodiments, the bandage 400 may be releasably disposed in an annular groove within the cavity 468 of the base portion 467. In other embodiments, the bandage 400 may be releasably adhered to the recess portion 466 of the cap 465. The recessed portion 466 may include a central opening 469 adapted for allowing the needle 425 to extend therethrough.

Prior to using the syringe 410, a health care worker or other user may place the cap 465 over the needle 425 such that the needle 425 extends into the cap 465. The needle 425 may pierce the bandage 400 or the bandage 400 may include an opening allowing the needle 425 to extend therethrough. The bandage 400 may be transferred from the cap 465 to the posterior end 423 of the hub 422. In some embodiments, the bandage 400 may include an adhesive coating in order to secure the bandage 400 to the posterior end 423 of the hub 422. In other embodiments, the posterior end 423 of the hub 422 may include an adhesive coating in order to secure the bandage 400 to the syringe 410. Therefore, the bandage 400 may be said to be loaded onto the syringe 410.

Additionally or alternatively, the cap 465 may provide a means of removing a cover (not shown) from the bandage 400. Thus, the bandage 400 may be disposed on the syringe 410 and the cap 465 may be placed over the needle 425 prior to using the syringe 410. The cap 465 may remove a covering or otherwise expose an adhesive coating on the outer surface of the bandage 400 opposite the syringe 410. The adhesive coating may allow the bandage 400 to be adhered to a patient's skin proximate a needle insertion point upon withdrawal of the needle 425 from the skin.

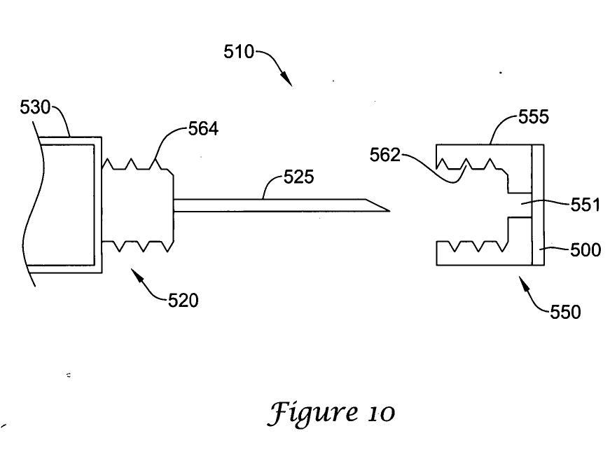

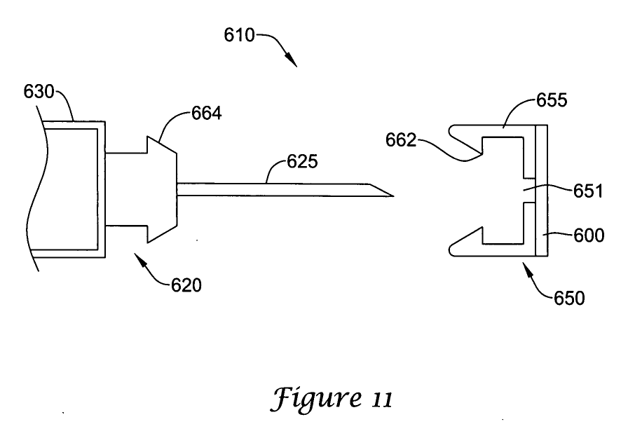

FIG. 10 shows a cartridge 550 including a bandage 500 configured to be coupled to a needle hub assembly 520 of a syringe 510. The bandage 500 may be substantially similar to other bandages previously disclosed herein, or the bandage 500 may be dissimilar. The needle hub assembly 520 may include a needle 525 and a threaded portion 564. In some embodiments, the threaded portion 564 may be a unitary or integral portion of the barrel 530 of the syringe 510. For instance, the threaded portion 564 may be formed in the posterior end of the barrel 530 during a molding process. In other embodiments, the threaded portion 564 may be a discrete portion secured to the barrel 530 of the syringe 510.

The cartridge 550 may include a body portion 555 having a threaded portion 562 configured to threadably engage with the threaded portion 564 of the needle hub assembly 520. Additionally, the cartridge 550 may include a central opening 551 allowing the needle 525 to pass therethrough. Upon extending through the central opening 551, the needle 525 may pierce the bandage 500 and extend beyond the bandage 500. Alternatively, the bandage 500 may have a central opening allowing the needle 525 to extend therethrough. The central opening of the bandage 500 may be axially aligned with the opening 551 of the cartridge 550. The bandage 500 may include an adhesive material, an absorbent material, an elastomeric material, a self-sealing material, a fibrous material, a gelatin substance, a liquidous substance, an ointment, or any combination or laminate of the same. Thus, the cartridge 550 may be removably disposed on and incorporated with the hub assembly 520 prior to administering an injection or letting of bodily fluids.

FIG. 11 shows an alternative cartridge 650 including a bandage 600. The bandage 600 may be substantially similar to other bandages previously disclosed herein, or the bandage 600 may be dissimilar. In many respects, the cartridge 650 may be similar to the cartridge 550 shown in FIG. 10. The cartridge 650 may be configured to be coupled to a needle hub assembly 620 of a syringe 610. The needle hub assembly 620 may include a needle 625 and an interlocking portion, such as a flanged portion 664. In some embodiments, the flanged portion 664 may be a unitary or integral portion of the barrel 630 of the syringe 610. In other embodiments, the flanged portion 664 may be a discrete portion secured to the barrel 630 of the syringe 610.

The cartridge 650 may include a body portion 655 having a complementary interlocking portion, such as a flanged portion 662, configured to mechanically interlock with the flanged portion 664 of the needle hub assembly 620. Additionally, the cartridge 650 may include a central opening 651 allowing the needle 625 to pass therethrough. Upon extending through the central opening 651, the needle 625 may pierce the bandage 600 and extend beyond the bandage 600. Alternatively, the bandage 600 may have a central opening allowing the needle 625 to extend therethrough. The central opening of the bandage 600 may be axially aligned with the opening 651 of the cartridge 650. The bandage 600 may include an adhesive material, an absorbent material, an elastomeric material, a self-sealing material, a fibrous material, a gelatin substance, a liquidous substance, an ointment, or any combination or laminate of the same. Thus, the cartridge 650 may be removably disposed on and incorporated with the hub assembly 620 prior to administering an injection or letting of bodily fluids.

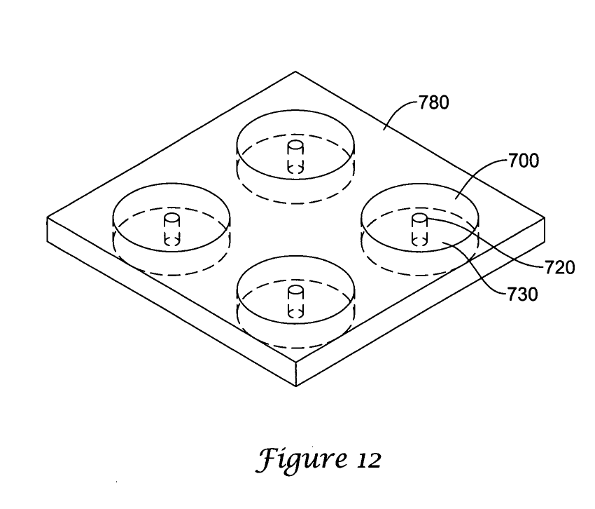

FIG. 12 shows a panel 780 for dispensing bandages 700. The bandages 700 may be substantially similar to other bandages previously disclosed herein, or the bandages 700 may be dissimilar. The panel 780 may include a plurality of bandages 700. For instance, the panel 780 may include an array of 4, 9, 10, 15, 20, 25, or more bandages 700. However, in other embodiments, the panel 780 may include any desired quantity of bandages 700. In some embodiments, the panel 780 may dispense cartridges, such as those cartridges described herein, including bandages 700 for coupling to a syringe. A health care worker or other user may load a bandage 700 onto the distal end of a syringe prior to use. Thus, the bandage 700 may be incorporated or integrated with a syringe prior to administering an injection or letting of a bodily fluid. The heath care worker or other user may pierce the central portion 720 of the bandage 700 with the needle of a syringe or otherwise extend a needle through the central portion 720 of the bandage 700. In some embodiments, the central portion 720 may be a through opening allowing a needle of a syringe to pass therethrough. The bandage 700 may be adhered to, coupled to, or otherwise temporarily secured to the syringe. The syringe may then be removed from the panel, thus removing the bandage 700 with the syringe. As the syringe including the bandage 700 is removed from the panel 780, the opposing side of the bandage 700 may be exposed, revealing an adhesive coating configured to adhere the bandage 700 to the skin of a patient upon withdrawal of the needle from a patient. In other embodiments, the bandage 700 may include a covering (not shown) which may be removed prior to using the syringe, revealing an adhesive coating on the posterior side of the bandage 700.

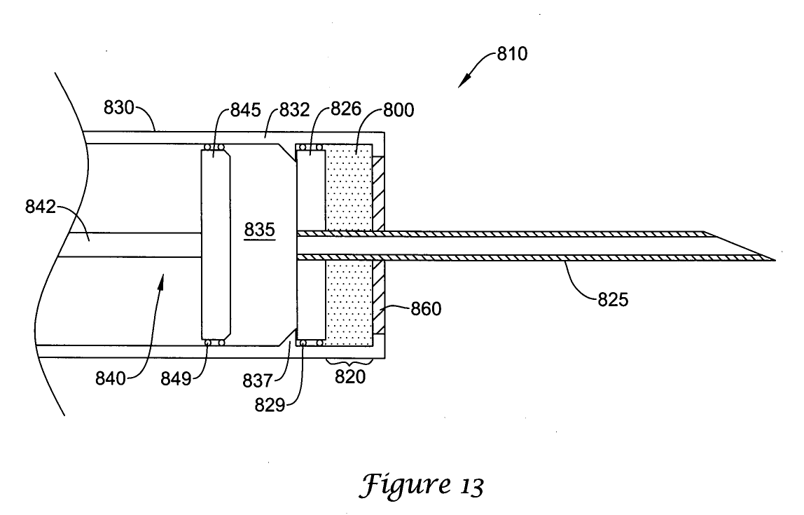

FIG. 13 shows an alternative syringe 810. The syringe 810 includes a barrel 830 having a tubular wall 832 defining a chamber 835. A plunger 840 including a plunger rod 842 attached to a piston 845 may be slidably disposed in the chamber 835 of the barrel 830. The piston 845 may include one or more resilient sealing members 849, such as annular 0-rings, for providing a fluid tight seal between the piston 845 and the inner surface 834 of the barrel 830.

The syringe 810 may include a needle 825 extending from the syringe 810. The needle 825 may be attached to a carrier 826. The carrier 826 may be slidably disposed within the barrel 830 of the syringe 810. One or more resilient sealing members 829 may be placed between the carrier 826 and the inner surface 834 of the barrel 830. The sealing member 829 may provide a fluid tight seal not allowing fluid to pass across the carrier 826. A lip 837 may extend around the inner surface 834 of the barrel 830 to prevent the carrier 826 from moving anteriorly (i.e. toward the plunger 840) within the syringe 810.

The syringe 810 may include a reservoir 820 containing a dressing substance 800, such as a liquidous or gelatin material, such as a hydropolymer adhesive dressing, a cyanoacrylate adhesive dressing, or other topical skin adhesive dressing. Alternatively the reservoir 820 may include an oxidized regenerated cellulose material, which may be topically administered to a needle insertion point to encourage hemostasis. The dressing substance 800 may arrest bleeding and/or provide a barrier against microorganisms at a needle insertion point on the skin of a patient. The reservoir 820 may be located posterior of the carrier 826, within the barrel 830 of the syringe 810. Thus, the carrier 826 may separate the reservoir 820 from the chamber 835. The sealing member 829 ensures fluid in the chamber 835 cannot enter the reservoir 820, and vise versa, thus fluidly separately the chamber 835 from the reservoir 820.

A membrane 860 may be located at the posterior end of the syringe 810. The membrane 860 may be configured to be breached, ruptured, permeated, or otherwise penetrated, when sufficient pressure is exerted on the membrane 860. In some embodiments, the membrane 860 may be a porous membrane selectively allowing a substance to pass therethrough. For example, when a sufficient pressure is exerted, the carrier 826 may expel the dressing substance 800 through openings in the porous membrane. In other embodiments, the membrane 860 may be sufficiently frangible such that the membrane 860 is ruptured when sufficient pressure is exerted on the membrane 860.

In operation, the plunger 840 may be actuated in the posterior direction (i.e. toward the needle 825) in order to expel medicinal or therapeutic fluid from the chamber 835 through the needle 825 and into a patient. At the point when the fluid in the chamber 835 is substantially completely expelled from the chamber 835, the piston 845 may contact the carrier 826 or otherwise trigger actuation of the carrier 826 in the posterior direction. As the carrier 826 is forced posteriorly, the carrier 826, in turn, forces the dressing substance 800 to breach, rupture, permeate, or otherwise penetrate the membrane 860. Exterior of the syringe 810, the dressing substance 800 may be topically applied to a needle insertion point on the skin of a patient prior to withdrawing the needle 825. As the needle 825 is withdrawn from the patient, the dressing substance 800 remains at the needle insertion point on the skin of the patient in order to arrest bleeding and/or provide a barrier against microorganisms at the wound.

Although various configurations, compositions, and designs of bandages and wound dressing materials and substances have been disclosed in detail, alternative bandages and wound dressing materials and substances are contemplated within the scope of the invention.

Although various concepts of the invention, have been presented in association with a hypodermic syringe, it is contemplated that concepts of the invention may be used in conjunction with other medical instruments including a needle, or the like, used in penetrating the skin of a body.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A syringe comprising:
a tubular barrel having an inner surface defining a fluid chamber, a first end and a second end;
a plunger movably disposed in the tubular barrel and extending from the first end of the tubular barrel;
a needle assembly coupled to the second end of the tubular barrel, the needle assembly including a needle and a needle hub; and
a wound dressing disposed on the needle hub.

2. The syringe of claim 1, wherein the wound dressing is a bandage.

3. The syringe of claim 2, wherein the bandage surrounds a portion of the needle.

4. The syringe of claim 2, wherein the needle extends through the bandage.

5. The syringe of claim 2, wherein the bandage includes a cylindrical disk having a first side, a second side, and a periphery surface.

6. The syringe of claim 5, wherein the first side of the bandage includes an adhesive.

7. The syringe of claim 6, wherein the second side of the bandage includes an adhesive.

8. The syringe of claim 7, wherein the adhesive on the first side of the bandage is a stronger adhesive than the adhesive on the second side of the bandage.

9. The syringe of claim 6, wherein the first side of the bandage includes a removable covering.

10. The syringe of claim 9, wherein the covering includes a tab to facilitate removing the covering from the bandage.

11. The syringe of claim 9, wherein the bandage includes a filament attached to the covering to facilitate removing the covering from the bandage.

12. The syringe of claim 2, wherein the bandage includes an annular ring of material having a central opening and an absorbent material disposed within the central opening of the ring.

13. The syringe of claim 12, wherein the needle extends through the central opening of the ring.

14. The syringe of claim 1, wherein the needle hub is threadedly coupled to the second end of the tubular barrel.

15. A syringe comprising:
a barrel;
a plunger disposed in the barrel;
a needle extending from one end of the barrel; and
a means for dressing a wound, the means for dressing a wound releasably integrated with the syringe.

16. The syringe of claim 15, wherein the means for dressing a wound includes an adhesive member.

17. The syringe of claim 15, wherein the means for dressing a wound includes an adhesive liquidous material.

18. The syringe of claim 15, wherein the means for dressing a wound includes a gelatin material.

19. A method of applying a bandage to the skin of a patient at a needle insertion point upon removal of a syringe, the method including the steps of:
providing a syringe including a needle and a bandage;
inserting the needle of the syringe into the skin of a patient at a needle insertion point;
actuating the syringe to transfer fluid;
adhering the bandage to the skin proximate the needle insertion point; and
removing the syringe after the step of adhering the bandage to the skin, thereby retaining the bandage at the needle insertion point.

20. The method of claim 19, wherein the needle extends through the bandage.

21. The method of claim 19, wherein the bandage includes an adhesive for adhering the bandage to the skin.

22. The method of claim 19, wherein during insertion of the needle, the bandage contacts the skin of the patient.
